(19) United States
(12) Patent Application Publication
(10) Pub. No.: US 2003/0092586 A1
(43) Pub. Date: May 15, 2003

(54) Composition

(75) Inventor: Subash Harmalker, Somerset, NJ (US)

Correspondence Address: COLGATE-PALMOLIVE COMPANY, 909 RIVER ROAD, PISCATAWAY, NJ, 08855, US

(73) Assignee: Colgate-Palmolive Company

(21) Appl. No.: 09/947,828

(22) Filed: Sep. 6, 2001

(51) Int. Cl. A61K 7/50

(52) U.S. Cl. 510/130; 510/480; 510/499; 510/382; 510/505

(57) ABSTRACT

In accordance with the invention, there is an aqueous fluid composition, which comprises:

a. cleansing effective amount of a surfactant or mixture thereof,

b. an antibacterial effective amount of a carbanilide compound or mixture thereof,

c. at least about 0.25 wt. % of composition of a compound or mixture thereof of the formula

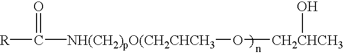

wherein R is alkyl or alkenyl of about 8 to about 20 carbon atoms, n is an integer of 1 to about 8, and p is 2 or 3.

d. and optionally an alcohol or mixture thereof of the formula

R′(CH₂CH₂O)_{q}H

where R′ is alkyl or alkenyl of 10 to 15 carbon atoms and q is 7, 8 or 9.

### BACKGROUND OF THE INVENTION

Fluid compositions capable of cleansing surfaces, including the skin, and also having antibacterial activity have been commercially available for many years. Generally, the criteria for a successful antibacterial in such a system is solubilization into the system, maintain stability of the system, precipitate out when water is added to the system and deposit on surface being treated while maintaining antibacterial efficacy.

Certain antibacterial agents satisfy these criteria relatively easily while others have varying degrees of difficulty meeting these criteria. One of the hardest to meet all these criteria in a fluid system are antibacterial agents that are members of the carbanilide family, more specifically triclocarban, hereafter referred to as TCC. It is quite difficult to solubilize TCC and then remove it from the system while having efficacy on the surface, as well as maintaining stability of the system during shelf life.

It has now been discovered that use of a specific solubilizer preferably together with a second solubilizer brings about a fluid cleansing system with a carbanilide antibacterial which is a stable system having a solubilized carbanilide, and which has antibacterial efficacy when precipitated onto a surface through the addition of water.

### SUMMARY OF THE INVENTION

In accordance with the invention, there is an aqueous fluid composition, which comprises:

a. a cleansing effective amount of a surfactant or mixture thereof,

b. an antibacterial effective amount of a carbanilide compound or mixture thereof,

c. at least about 0.25 wt. % of composition of a compound or mixture thereof of the formula

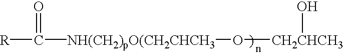

wherein R is alkyl or alkenyl of about 8 to about 20 carbon atoms, n is an integer of 1 to about 8, and p is 2 or 3.

A further aspect of the invention is an aqueous, fluid composition, which comprises:

a. at least about 5 wt. % of a surfactant or mixture thereof,

b. at least about 0.03 wt. % of an antibacterial carbanilide or mixture thereof,

c. at least about 0.25 wt. % of composition of a compound or mixture thereof of the formula

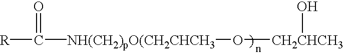

wherein R is alkyl or alkenyl of about 8 to about 20 carbon atoms, n is an integer of 1 to about 8, and p is 2 or 3.

Another aspect of the invention is applying a composition as identified above after or concomitantly with dilution with water to a surface, particularly skin, and obtaining a combination of surface cleansing and antibacterial efficacy.

A still further aspect of the invention is a fluid system of a fragrance, a halogenated carbanilide, a compound or mixture thereof of the formula

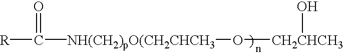

wherein R is alkyl or alkenyl of about 8 to about 20 carbon atoms, n is an integer of 1 to about 8, and p is 2 or 3, and, optionally, an alcohol or mixture thereof of the formula,

R′(CH₂CH₂O)_{q}H

wherein R′ is alkyl or alkenyl of 10 to 15 carbon atoms and q is 7, 8 or 9.

### DETAILED DESCRIPTION OF THE INVENTION

The aqueous fluid system is generally a liquid or a gel. Such system should remain the same visually with respect to clarity, separation, precipitate and the like with the surfactant, the antibacterial agents and stabilizing solubilizing material therein over a specified period of time to be called stable in comparison to the composition without the stabilizing, solubilizing material therein.

The surface to be cleansed can be any hard surface such as a sink, toilet, countertop and dish found in the home, or a person's skin. Personal care products for skin cleansing are preferred.

Any surfactant or mixture of surfactants at an effective amount for cleansing can be employed.

The surfactant can be anionic, nonionic, amphoteric, or cationic, preferably anionic. Soap, a long chain alkyl or alkenyl, branched or normal carboxylic acid salt such as sodium, potassium, ammonium or substituted ammonium salt can be present in the composition as an example of an anionic surfactant. Exemplary of long chain alkyl or alkenyl are from about 8 to about 22 carbon atoms in length, specifically about 10 to about 20 carbon atoms in length, more specifically alkyl and most specifically normal, or normal with little branching. Small quantities of olefinic bond(s) may be present in the predominantly alkyl sections, particularly if the source of the “alkyl” group is obtained from a natural product such as tallow, coconut oil and the like. Because of its potential harshness soap is not a preferred surfactant and can be omitted from the composition.

Other surfactants can be present in the composition as well. Examples of such surfactants are the anionic, amphoteric, nonionic and cationic surfactants. Examples of anionic surfactants include but are not limited to soaps, alkyl sulfates, anionic acyl sarcosinates, methyl acyl taurates, N-acyl glutamates, acyl isethionates, alkyl sulfosuccinates, alkyl phosphate esters, ethoxylated alkyl phosphate esters, trideceth sulfates, protein condensates, mixtures of ethoxylated alkyl sulfates and the like.

Alkyl chains for these surfactants are C₈-C₂₂, preferably C₁₀-C₁₈, more preferably C₁₂-C₁₄.

Anionic non-soap surfactants can be exemplified by the alkali metal salts of organic sulfate having in their molecular structure an alkyl radical containing from about 8 to about 22 carbon atoms and a sulfonic acid or sulfuric acid ester radical (included in the term alkyl is the alkyl portion of higher acyl radicals). Preferred are the sodium, ammonium, potassium or triethanolamine alkyl sulfates, especially those obtained by sulfating the higher alcohols (C₈-C₁₈ carbon atoms), sodium coconut oil fatty acid monoglyceride sulfates and sulfonates; sodium or potassium salts of sulfuric acid esters of the reaction product of 1 mole of a higher fatty alcohol (e.g., tallow or coconut oil alcohols) and 1 to 12 moles of ethylene oxide; sodium or potassium salts of alkyl phenol ethylene oxide ether sulfate with 1 to 10 units of ethylene oxide per molecule and in which the alkyl radicals contain from 8 to 12 carbon atoms, sodium alkyl glyceryl ether sulfonates; the reaction product of fatty acids having from 10 to 22 carbon atoms esterified with isethionic acid and neutralized with sodium hydroxide; water soluble salts of condensation products of fatty acids with sarcosine; and others known in the art.

Zwitterionic surfactants can be exemplified by those which can be broadly described as derivatives of aliphatic quaternary ammonium, phosphonium, and sulfonium compounds, in which the aliphatic radicals can be straight chain or branched and wherein one of the aliphatic substituents contains from about 8 to 18 carbon atoms and one contains an anionic water-solubilizing group, e.g., carboxy, sulfonate, sulfate, phosphate, or phosphonate. A general formula for these compounds is:

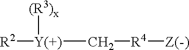

wherein R² contains an alkyl, alkenyl, or hydroxy alkyl radical of from about 8 to about 18 carbon atoms, from 0 to about 10 ethylene oxide moieties and from 0 to 1 glyceryl moiety; Y is selected from the group consisting of nitrogen, phosphorus, and sulfur atoms; R3 is an alkyl or monohydroxyalkyl group containing 1 to about 3 carbon atoms; X is 1 when Y is a sulfur atom and 2 when Y is a nitrogen or phosphorus atom, R⁴ is an alkylene or hydroxyalkylene of from 0 to about 4 carbon atoms and Z is a radical selected from the group consisting of carboxylate, sulfonate, sulfate, phosphonate, and phosphate groups.

Examples include: 4-[N,N-di(2-hydroxyethyl)-N-octadecylammonio]-butane-1-carboxylate; 5-[S-3-hydroxypropyl-S-hexadecylsulfonio]-3 hydroxypentane-1-sulfate; 3-[P,P-P-diethyl-P 3,6,9 trioxatetradecyl-phosphonio]-2-hydroxypropane-1-phosphate; 3-[N,N-dipropyl-N-3 dodecoxy-2-hydroxypropylammonio]-propane-1-phosphonate; 3-(N,N-di-methyl-N-hexadecylammonio) propane-1-sulfonate; 3-(N,N-dimethyl-N-hexadecylammonio)-2-hydroxypropane-1-sulfonate; 4-(N,N-di(2-hydroxyethyl)-N-(2 hydroxydodecyl)ammonio]-butane-1-carboxylate; 3-[S-ethyl-S-(3-dodecoxy-2-hydroxypropyl)sulfonio]-propane-1-phosphate; 3-(P,P-dimethyl-P-dodecylphosphonio)-propane-1-phosphonate; and 5-[N,N-di(3-hydroxypropyl)-N-hexadecylammonio]-2-hydroxy-pentane-1-sulfate.

Examples of amphoteric surfactants which can be used in the compositions of the present invention are those which can be broadly described as derivatives of aliphatic secondary and tertiary amines in which the aliphatic radical can be straight chain or branched and wherein one of the aliphatic substituents contains from about 8 to about 18 carbon atoms and one contains an anionic water solubilizing group, e.g., carboxy, sulfonate, sulfate, phosphate, or phosphonate. Examples of compounds falling within this definition are sodium 3-dodecylaminopropionate, sodium 3-dodecylaminopropane sulfonate, N-alkyltaurines, such as the one prepared by reacting dodecylamine with sodium isethionate according to the teaching of U.S. Pat. No. 2,658,072, N-higher alkyl aspartic acids, such as those produced according to the teaching of U.S. Pat. No. 2,438,091, and the products sold under the trade name “Miranol” and described in U.S. Pat. No. 2,528,378. Other amphoterics such as betaines are also useful in the present composition.

Examples of betaines useful herein include the high alkyl betaines such as coco dimethyl carboxymethyl betaine, lauryl dimethyl carboxy-methyl betaine, lauryl dimethyl alpha-carboxyethyl betaine, cetyl dimethyl carboxymethyl betaine, lauryl bis-(2-hydroxyethyl)carboxy methyl betaine, stearyl bis-(2-hydroxypropyl) carboxymethyl betaine, oleyl dimethyl gamma-carboxypropyl betaine, lauryl bis-(2-hydro-xypropyl) alpha-carboxyethyl betaine, etc. The sulfobetaines may be represented by coco dimethyl sulfopropyl betaine, stearyl dimethyl sulfopropyl betaine, amido betaines, amidosulfobetaines, and the like.

Many cationic surfactants are known to the art. By way of example, the following may be mentioned:

stearyldimenthylbenzyl ammonium chloride;

dodecyltrimethylammonium chloride;

nonylbenzylethyldimethyl ammonium nitrate;

tetradecylpyridinium bromide;

laurylpyridinium chloride;

cetylpyridinium chloride

laurylpyridinium chloride;

laurylisoquinolium bromide;

ditallow(Hydrogenated)dimethyl ammonium chloride;

dilauryldimethyl ammonium chloride; and

stearalkonium chloride.

Additional cationic surfactants are disclosed in U.S. Pat. No. 4,303,543 see column 4, lines 58 and column 5, lines 1-42, incorporated herein by references. Also see CTFA Cosmetic Ingredient Dictionary, 4th Edition 1991, pages 509-514 for various long chain alkyl cationic surfactants; incorporated herein by references.

Nonionic surfactants can be broadly defined as compounds produced by the condensation of alkylene oxide groups (hydrophilic in nature) with an organic hydrophobic compound, which may be aliphatic or alkyl aromatic in nature. Examples of preferred classes of nonionic surfactants are:

1. The polyethylene oxide condensates of alkyl phenols, e.g., the condensation products of alkyl phenols having an alkyl group containing from about 6 to 12 carbon atoms in either a straight chain or branched chain configuration, with ethylene oxide, the said ethylene oxide being present in amounts equal to 10 to 60 moles of ethylene oxide per mole of alkyl phenol. The alkyl substituent in such compounds may be derived from polymerized propylene, diisobutylene, octane, or nonane, for example.

2. Those derived from the condensation of ethylene oxide with the product resulting from the reaction of propylene oxide and ethylene diamine products which may be varied in composition depending upon the balance between the hydrophobic and hydrophilic elements which is desired. For example, compounds containing from about 40% to about 80% polyoxyethylene by weight and having a molecular weight of from about 5,000 to about 11,000 resulting from the reaction of ethylene oxide groups with a hydrophobic base constituted of the reaction product of ethylene diamine and excess propylene oxide, said base having a molecular weight of the order of 2,500 to 3,000, are satisfactory.

3. The condensation product of aliphatic alcohols having from 8 to 18 carbon atoms, in either straight chain or branched chain configuration with ethylene oxide, e.g., a coconut alcohol ethylene oxide condensate having from 10 to 30 moles of ethylene oxide per mole of coconut alcohol, the coconut alcohol fraction having from 10 to 14 carbon atoms. Other ethylene oxide condensation products are ethoxylated fatty acid esters of polyhydric alcohols (e.g., Tween 20-polyoxyethylene (20) sorbitan monolaurate).

4. Long chain tertiary amine oxides corresponding to the following general formula:

R₁R₂R₃N→O

wherein R₁ contains an alkyl, alkenyl or monohydroxy alkyl radical of from about 8 to about 18 carbon atoms, from 0 to about 10 ethylene oxide moieties, and from 0 to 1 glyceryl moiety, and, R₂ and R₃ contain from 1 to about 3 carbon atoms and from 0 to about 1 hydroxy group, e.g., methyl, ethyl, propyl, hydroxy ethyl, or hydroxy propyl radicals. The arrow in the formula is a conventional representation of a semipolar bond. Examples of amine oxides suitable for use in this invention include dimethyldodecylamine oxide, oleyl-di(2-hydroxyethyl)amine oxide, dimethyloctylamine oxide, dimethyldecylamine oxide, dimethyltetradecylamine oxide, 3,6,9 trioxaheptadecyldiethylamine oxide, di(2-hydroxyethyl)-tetradecylamine oxide, 2-dodecoxyethyldimethylamine oxide, 3-dodecoxy-2-hydroxypropyldi(3-hydroxypropyl)amine oxide, dimethylhexadecylamine oxide.

5. Long chain tertiary phosphine oxides corresponding to the following general formula:

RR′R″P→O

wherein R contains an alkyl, alkenyl or monohydroxyalkyl radical ranging from 8 to 20 carbon atoms in chain length, from 0 to about 10 ethylene oxide moieties and from 0 to 1 glyceryl moiety and R′ and R″ are each alkyl or monohydroxyalkyl groups containing from 1 to 3 carbon atoms. The arrow in the formula is a conventional representation of a semipolar bond. Examples of suitable phosphine oxides are: dodecyldimethylphosphine oxide, tetradecylmethylethylphosphine oxide, 3,6,9-trioxaoctadecyldimethylphosphine oxide, cetyldimethylphosphine oxide, 3-dodecoxy-2-hydroxypropyldi(2-hydroxyethyl) phosphine oxide stearyldimethylphosphine oxide, cetylethyl propylphosphine oxide, oleyldiethylphosphine oxide, dodecyldiethylphosphine oxide, tetradecyldiethylphosphine oxide, dodecyldipropylphosphine oxide, dodecyldi(hydroxymethyl)phosphine oxide, dodecyldi(2-hydroxyethyl)phosphine oxide, tetradecylmethyl-2-hydroxypropylphosphine oxide, oleyldimethylphosphine oxide, 2-hydroxydodecyldimethylphosphine oxide.

6. Long chain dialkyl sulfoxides containing one short chain alkyl or hydroxy alkyl radical of 1 to about 3 carbon atoms (usually methyl) and one long hydrophobic chain which contain alkyl, alkenyl, hydroxy alkyl, or keto alkyl radicals containing from about 8 to about 20 carbon atoms, from 0 to about 10 ethylene oxide moieties and from 0 to 1 glyceryl moiety. Examples include: octadecyl methyl sulfoxide, 2-ketotridecyl methyl sulfoxide, 3,6,9-trioxaoctadecyl 2-hydroxyethyl sulfoxide, dodecyl methyl sulfoxide, oleyl 3-hydroxypropyl sulfoxide, tetradecyl methyl sulfoxide, 3 methoxytridecylmethyl sulfoxide, 3-hydroxytridecyl methyl sulfoxide, 3-hydroxy-4-dodecoxybutyl methyl sulfoxide.

7. Alkylated polyglycosides wherein the alkyl group is from about 8 to about 20 carbon atoms, preferably about 10 to about 18 carbon atoms and the degree of polymerization of the glycoside is from about 1 to about 3, preferably about 1.3 to about 2.0.

The antibacterial agent employed in the invention is a member of the carbanilide family. Illustrative examples of the carbanilide family include carbanilide per se; 3′-trifluoromethyl-4,4′dichlorocarbanilide,3,3′,4′-trichlorocarbanilide, and the like. Most preferred is the halogenated carbanilide known as triclocarban, also known as 3,4,4′-trichlorocarbanilide.

The agent(s) which bring about the aforementioned desired characteristics of the formulation, solubilization of the antibacterial carbanilide, stability of the solubilized carbanilide containing formulation, and upon water dilution the deposition of the efficacious antibacterial upon the surface, particularly skin, is an effective amount of the antibacterial compound or mixture thereof of compound(s) of the formula:

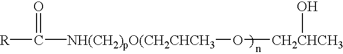

wherein R is alkyl or alkenyl of about 8 to 20 carbon atoms and mixture thereof, p is 2 or 3, and n is an integer of 1 to about 8. Examples of R groups, both branched and normal include octyl, decyl, isodecyl, 2,2,4 trimethylpentyl-4-2-ethyl decyl, dodecyl, myristyl, stearyl, palmityl, eicosyl, isostearyl, and the like including cocoyl and other naturally occurring mixtures as well as corresponding alkenyl compounds. The integer n can be 1, 2, 3, 4, 5, 6, 7 or 8, more preferred n is 1, 2, 3, or 4 and most preferred is n is 1. p is preferably 2. A preferred R is alkyl and/or alkenyl of about 12 to about 16 carbon atoms, preferably a mixture such as cocoyl. The most preferred compound is PPG-2 hydroxyethyl cocoamide, wherein R is cocoyl, p is 2, and n is 1, available from Mona Industries as Promidium CO.

It is preferred to use a cosolubilizer with the above amides. This cosolubilizer is a long chain ethoxylated primary alcohol of the formula,

R′(CH₂CH₂O)_{q}H

wherein R₁ is alkyl and/or alkenyl, preferably alkyl, of about ten to fifteen carbon atoms and q is an integer of 7, 8, or 9, preferably 7. The most preferred compound is laureth-7. The use of the cosolubilizer allows the combination of solubilizers to bring about dissolving of TCC in a formulation at room temperature. With the amide alone, heat must be applied to the formulation to bring about proper dissolving of the carbanilides. Chlorinated carbanilides, TCC in particular, upon heating can breakdown into chloroanilines, a particular undesired breakdown product because of its potential carcinogenicity. Therefore, the ability to process chlorinated carbanilides at room temperature is a distinct advantage.

The quantity of surfactant employed is any quantity that brings about cleansing of any of the aforementioned surfaces, particularly skin. At least about 1 wt. %, desirably about 2, 3, 4, 5, 6, 7, or 8 wt. % of a surfactant or mixture of surfactants can be employed in the composition. Generally, at least about 20 wt. % of the surfactant present is anionic surfactant. The maximum amount of surfactant is not unduly significant and is generally a matter of other characteristics of the composition and cost. Generally, a composition having less than about 30, 25, 20, 15 or 12 wt. % of surfactant or mixture is desirable. Generally, compositions having any surfactants such as anionics for example, sodium laureth sulfate (SLES), alpha olefin sulfonate (AOS), betaines such as cocoamidopropyl betaine (CAPB) and nonionic such as alkyl polyglucoside (APG) therein are preferred. In calculating the weight of surfactant therein, neither the solubilizer nor cosolubilizer of this invention is counted in the surfactant total.

With respect to the carbanilides, particularly TCC, any antibacterial effective amount can be employed in the composition. Generally, at least about 0.01 wt. %, desirably above about 0.02, 0.03, 0.04 and 0.05 will bring about good results. Generally, no more than about 1.0, 0.9, 0.75 wt. % or 0.50 carbanilide is employed. The upper limit is at least somewhat dependent on the solubility of the carbanilide in the composition.

The solubilizing amide group of compound is present at quantities sufficient to bring about the above identified desirable properties. Generally, a minimum amount is about 0.25, 0.33, 0.4 or 0.5 wt. % of the composition. A maximum amount is determined by its effect on other characteristics of the formulation and cost but is generally no more than about 5, 4, or 3 wt. % of the composition.

The cosolubilizing long chain ethoxylated alcohol is present at quantities sufficient to assist in the achievement of the desirable formulation properties. Generally, a minimum amount is about 0.25, 0.33, 0.4 or 0.5 wt. % of the composition. A maximum amount is determined by its effect on the characteristics of the formulation and cost but is generally no more than about 5, 4 or 3 wt. % of the composition. When used together, the amide is preferably about 0.3 to 3 times the weight of the alcohol, more preferably about 0.7 to 1.3.

It has been found that the carbanilide, particularly a halogenated carbanilide can be readily processed into the final fluid system by adding it to a fragrance when employed in the final fluid system. Fragrance(s) heavily organic in nature and comprising various odor causing components including oftentime one or more phenolic compounds make an excellent solubilizing system for the carbanilide (preferably halogenated), the amide solubilizing component, and, if employed, the cosolubilizing long chain ethoxy alcohol. Such mixture is made at room temperature and does not ordinarily require warming so as to significantly reduce the quantity of chloroanilines produced from chlorinated carbanilides under hydrolyzing conditions. Additionally, it is believed that the presence of the fragrance in combination with the amide assists in reducing the quantity of chloroanilines produced from hydrolyzing conditions of halogenated carbanilides, particularly TCC. In this composition of fragrance, carbanilide, amide and optionally alcohol, the approximate quantities are 5-60 wt. %, 1-20 wt. %, 1-85 wt. % and 0-75 wt. %, respectively.

Various other material can be present in the composition including emollients, colorants, thickeners, anti-inflammatories, chelators, and the like.

The fluid systems of this invention can be prepared in the following manner.

A. Sodium Laureth Sulfate (SLES) Based Shower Gel

1. Prepare TCC Premix (Promidium Co, Laureth-7, Perfume and TCC) in a separate premix tank. Follow the order of addition to obtain clear solution at room temperature. It is preferable to not heat the solution to avoid or minimize the formation of chloroanilines.

Order or Addition

Promidium Co

Laureth-7

Perfume

TCC

Mix thoroughly to obtain a clear solution.

If TCC as added to Promidium Co before the latter is blended with Laureth-7 and Perfume, it will be difficult to get a clear solution at room temperature.

2. Add deionized water to the main tank

3. Add to the deionized water

EDTA

Dyes

Maintain agitation at low speed, Ensure at least five minutes dispersion time between each ingredient.

4. In a separate premix tank, dissolve a Polyquaternium, preferably Polyquat 7, if employed, with deionized water (ratio 50/50) and transfer the solution to the main tank.

5. Add Coco Amido Propyl Betaine to the main tank under slight agitation.

6. Add SLES under slight agitation. Avoid or minimize foam formation by controlling agitation speed.

7. Add alkyl polyglycoside heated to 40-50° C. under slight agitation.

8. Add TCC premix and increase the agitation speed till the whole solution is homogeneous.

9. Adjust pH to the specifications with an organic acid such as citric acid solution.

10. Add preservative under agitation to ensure thorough mixing.

11. Adjust viscosity to the specifications with viscosity modifying agent such as sodium chloride solution.

B. Alpha Olefin Sulfonate Based Shower Gel

1. Prepare TCC Premix (Promidium Co, Laureth-7, Perfume and TCC) in a separate premix tank. Follow the order to addition strictly, to obtain clear solution at room temperature. It is preferable to not heat the solution to avoid formation of chloroanilines.

Order of Addition

Promidium Co

Laureth-7

Perfume

TCC

Mix thoroughly to obtain a clear solution.

If TCC is added to Promidium Co before the latter is blended with Laureth-7 and Perfume, it will be very difficult to get a clear solution at room temperature.

2. Add deionized water to the main tank.

3. Add to the deionized water add EDTA, dyes, Glycerine Aloe Vera Gel and Silk Proteins.

Maintain agitation at low speed. Ensure five minutes dispersion time between each ingredient.

4. In a separate premix tank, dissolve Polyquaternium, preferably Polyquat 7, if employed with deionized water (ratio 50/50) and transfer the solution to the main tank.

5. Add AOS (Alpha Olefin Sulfonate) under slight agitation. Avoid foam formation by controlling agitation speed.

6. Add Coco Amido Propyl Betaine to the main tank under slight agitation.

7. Add TCC premix and increase the agitation speed till the whole solution is homogeneous.

8. Adjust pH to the specifications with citric acid solution.

9. Add preservative under agitation to ensure thorough mixing.

10. Adjust viscosity to the specifications with sodium chloride solution.

The following are examples of the invention. These are illustrative of the invention scope.

### EXAMPLE 1

| Ingredient Name | Weight % |
|---|---|
| Sodium Laureth Sulfate | 8.2 |
| Cocamidopropyl Betaine | 3.0 |
| Lauryl Polyglucose | 1.1 |
| Perfume | 1.0 |
| Sodium Chloride | 0.9 |
| Laureth Alcohol | 0.5 |
| Laureth-7 | 0.3 |
| PPG-2 Hydroxyethyl Cocamide | 0.3 |
| Sodium Sulfate | 0.3 |
| Polyquaternium-7 | 0.2 |
| Glycerin | 0.1 |
| Tetrasodium EDTA | 0.08 |
| Triclocarban | 0.075 |
| Citric Acid | 0.04 |
| Preservatives, etc. | 0.27 |
| Water | Q.S. |
| TOTAL | 100 |

### EXAMPLE 2

| Ingredient Name | | Weight % |
|---|---|---|
| Sodium Laureth Sulfate | | 8.2 |
| Cocamidopropyl Betaine | | 3.2 |
| Lauryl Polyglucose | | 1.1 |
| Perfume | | 1.0 |
| Glycol Distearate | | 0.9 |
| Laureth-7 | | 0.7 |
| PPG-2 Hydroxyethyl Cocamide | | 0.7 |
| Sodium Chloride | | 0.6 |
| Laureth-4 | | 0.5 |
| Laureth Alcohol | | 0.4 |
| Sodium Sulfate | | 0.3 |
| Polyquaternium-7 | | 0.2 |
| Triclocarban | | 0.15 |
| Tetrasodium EDTA | | |
| Citric Acid | {close oversize brace} | 0.4 |
| Formic Acid | | |
| Preservatives, etc. | | |
| Water | | Q.S. |
| TOTAL | | 100 |

### EXAMPLE 3

| Ingredient Name | | Weight % |
|---|---|---|
| Sodium C₁₄₋₁₆ Olefin Sulfonate | | 8.1 |
| Cocamidopropyl Betaine | | 2.4 |
| PPG-2 Hydroxyethyl Cocamide | | 2.0 |
| Sodium Chloride | | 1.8 |
| Laureth-7 | | 1.0 |
| Perfume | | 0.4 |
| Citric Acid | | 0.3 |
| Sodium Sulfate | | 0.3 |
| Triclocarban | | 0.15 |
| Tetrasodium EDTA | | |
| Polyquaternium-7 | {close oversize brace} | 0.4 |
| Preservatives, etc. | | |
| Water | | Q.S. |
| TOTAL | | 100 |

### EXAMPLE 4

Using the formulation of Example 1 or Example 2 but with varying amounts of TCC, the amide wherein R is cocoyl, p is 2 and n is 1, and the alcohol wherein R′ is lauryl and q is 7, antibacterial efficacy is measured using a cup scrub method on humans against the composition without TCC. The presence of Promidium Co and/or laureth-7 does not significantly affect antibacterial efficacy.

| | | Wt. % | | | Antibacterial | Antibacterial |
|---|---|---|---|---|---|---|
| | Wt. % | Promidium | Wt. % | Wt. % | Efficacy, Log | Efficacy, |
| Composition | TCC | Co | Laureth-7 | Fragrance | Reduction | Hours |
| Example 1 | 0.075 | 0.34 | 0.34 | 1 | >1 | 8 |
| Example 2 | 0.15 | 0.67 | 0.67 | 1 | >1 | 24 |
| Example 1 | 0.225 | 1.01 | 1.01 | 1 | >1 | 24 |

As shown by this data, TCC brings about a greater than 1 log reduction of bacteria as measured against a composition without TCC.

1. An aqueous fluid composition, which comprises:
a. cleansing effective amount of a surfactant or mixture thereof,
b. an antibacterial effective amount of a carbanilide compound or mixture thereof,
c. at least about 0.25 wt. % of composition of a compound or mixture thereof of the formula 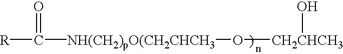
wherein r is alkyl or alkenyl of about 8 to about 20 carbon atoms, n is an integer of 1 to about 8, and p is 2 or 3

2. The composition in accordance with claim 1 wherein the carbanilide is TCC.

3. The composition in accordance with claim 1 wherein in component c, p is 2 and n is 1.

4. The composition in accordance with claim 1 wherein additionally present is an alcohol compound or mixture of the formula
R′(CH₂CH₂O)_{q}H
wherein R′ is alkyl or alkenyl of 10 to 15 carbon atoms and q is 7 to 9.

5. The composition in accordance with claim 3 wherein R is cocoyl.

6. The composition in accordance with claim 4 wherein R′ is lauryl and q is 7.

7. The composition in accordance with claim 6 wherein
a. the carbanilide is TCC, and
b. in component c, R is about 12 to 16 carbon atoms, p is 2 and n is 1.

8. The composition in accordance with claim 7 wherein
a. surfactant or mixture thereof is at least about 5 wt. %,
b. TCC is at least about 0.03 wt. %
c. Component c of claim 1 wherein R is cocoyl is at least about 0.25 wt. %, and
d. the alcohol is at least about 0.25 wt. %.

9. A method of applying to a surface the composition of claim 1 after or concomitantly with dilution by water and obtaining a combination of surface cleansing and antibacterial efficacy.

10. The method in accordance with claim 9 wherein the surface is skin.

11. A method of applying to a surface the composition of claim 8 after or concomitantly with dilution by water and obtaining a combination of surface cleansing and antibacterial efficacy.

12. The method in accordance with claim 1 wherein the surface is skin.

13. A composition comprising
a. a fragrance
b. an antibacterial carbanilide
c. a compound or mixture thereof of the formula 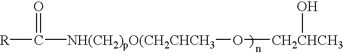
wherein r is alkyl or alkenyl of about 8 to about 20 carbon atoms, n is an integer of 1 to about 8, and p is 2 or 3.

14. The composition in accordance with claim 13 wherein the carbanilide is TCC.

15. The composition in accordance with claim 13 wherein n is 1 and p is 2.

16. The composition in accordance with claim 13 wherein additionally present is an alcohol compound or mixture thereof of the formula
R′(CH₂CH₂O)_{q}H
wherein R′ is alkyl or alkenyl of 10 to 15 carbon atoms, and q is 7, 8 or 9.

17. The combination in accordance with claim 16 wherein
a. the carbanilide is TCC
b. R is about 10 to 14 carbon atoms, n is 1 and p is 2
c. R′ is lauryl and q is 7.
